(19) United States
(12) Patent Application Publication
(10) Pub. No.: US 2024/0238030 A1
(43) Pub. Date: Jul. 18, 2024

(54) ABLATION DEVICE FOR CONTROLLED TEAR PROPAGATION IN GENERATION OF MULTI-LEAFLET INTERATRIAL SHUNT

(71) Applicant: Medtronic, Inc., Minneapolis, MN (US)

(72) Inventors: Neal C. Duong, Ramsey, MN (US); Nicolas Coulombe, Anjou (CA)

(21) Appl. No.: 18/408,299

(22) Filed: Jan. 9, 2024

Related U.S. Application Data
(60) Provisional application No. 63/479,669, filed on Jan. 12, 2023.

(51) Int. Cl. A61B 18/02; A61B 18/14

(52) U.S. Cl. CPC A61B 18/0218; A61B 18/1492; A61B 2017/00252

(57) ABSTRACT

Example devices and techniques are described herein for creating a multicuspid, multi-leaf interatrial shunt in the heart of a patient. An example device includes an elongate tool body and an inflatable balloon. The elongate tool body includes at least one lumen and a distal portion. The inflatable balloon is on the distal portion, and is configured to be inflated via the lumen. The inflatable balloon includes a distal outer surface configured for contacting an interatrial septum of a heart of a patient, and an ablation region of the distal outer surface of the inflatable balloon configured for ablating the interatrial septum when the inflatable balloon receives the ablation energy. The ablation region is disposed on the distal outer surface of the inflatable balloon in a manner that the ablation region creates a multicuspid ablation pattern when the inflatable balloon receives the ablation energy.

This application claims the benefit of U.S. Provisional Application No. 63/479,669 filed Jan. 12, 2023, the entire contents of which is incorporated herein by reference.

### TECHNICAL FIELD

This disclosure generally relates to medical devices, and more particularly, to medical devices and associated techniques for forming shunts.

### BACKGROUND

Heart failure is a common syndrome in which a patient's heart output is insufficient to meet the body's needs. In some forms of heart failure, excess pressure can occur in the left atrium, causing a variety of symptoms. Patients experiencing heart failure currently have limited treatment options.

Interatrial shunting is a technique to decompress the left atrium in patients suffering from heart failure. During the procedure, a blood flow pathway is created between the right atrium and the left atrium such that blood flows between them. In a typical procedure, the septal wall separating the atria is cut with a puncturing device and a mechanical device such as a stent is left in place to prevent tissue overgrowth and to maintain the shunt.

### SUMMARY

The present disclosure describes systems, devices, and techniques for creating a fluid pathway, or shunt, between the left atrium and right atrium of a heart of a patient. The shunt can be used, for example, to treat patients with heart failure. Many existing interatrial shunting procedures implant a device into the interatrial septum to maintain such a shunt. Some other interatrial shunting procedures forgo an implant and rely on a cutting tool to create a controlled opening within the interatrial septum, and a cryoablation device to ensure myocardial cell death to prevent regrowth. These avoid a permanent implant in the heart, reducing risk of failure and its potential prevention from future trans-septal catheter access.

Some examples described herein employ a cryoablation balloon. The distal outer surface of the cryoablation balloon, which contacts the interatrial septum, has an insulated region and a cooling region (e.g., a non-insulated region). The insulated region has a low thermal conductivity to protect the tissue from the cooling of a cryogenic agent in the cryoablation balloon. The cooling region is configured to have a specified shape to apply an ablated profile in the tissue. That is, the cooling region kills the cells of the tissue of the interatrial septum it is in contact with, altering the mechanical properties of that region of the septum. This cryo-ablated tissue has a significantly lower tensile strength compared to untreated regions of the interatrial septum. Thus, the ablated tissue is primed for a controlled tearing as the balloon dilates a puncture opening.

After performing the initial ablation with the distal outer surface of the balloon to leave the desired ablation profile, the balloon is deflated and crossed partially through a puncture in the ablated profile from the right atrium into the left atrium. The balloon may then be inflated with a non-cryogenic agent (e.g., air, nitrous oxide, etc.) in a controlled manner to dilate the opening through which the balloon passed to a desired size. Here, because the ablated tissue has lower tensile strength, there is a higher likelihood for a tear propagation to occur along the arms of the ablated tissue profile. Accordingly, a multicuspid, multi-leaflet interatrial shunt is created between the left and right atria to treat heart failure.

In one example, a surgical apparatus includes an elongate tool body having at least one lumen and a distal portion. The surgical apparatus further includes an inflatable balloon on the distal portion, the inflatable balloon configured to be inflated via the lumen. The inflatable balloon includes a distal outer surface configured for contacting an interatrial septum of a heart of a patient, and an ablation region of the distal outer surface of the inflatable balloon for extracting heat from the interatrial septum when the interior chamber of the balloon receives the cryogenic agent. The ablation region is disposed on the distal outer surface of the inflatable balloon in a manner that the ablation region creates a multicuspid ablation pattern when the inflatable balloon receives the ablation energy.

In another example, a surgical apparatus includes an elongate tool body having at least one lumen and a distal portion. The elongate tool body includes a distal puncturing tip for puncturing an interatrial septum of a heart of a patient to create an opening, and an inflatable balloon configured to be inflated via the lumen of the elongate tool body. The inflatable balloon includes a distal outer surface configured for contacting the interatrial septum, and an ablation region of the distal outer surface of the inflatable balloon for ablating the interatrial septum when the inflatable balloon receives the ablation energy. The ablation region is disposed on the distal outer surface of the inflatable balloon in a manner that the ablation region creates a multicuspid ablation pattern when the inflatable balloon receives the ablation energy. The inflatable balloon is configured to advance through the opening and to dilate the opening causing tissue to tear along the multicuspid ablation pattern.

In another example, a method includes creating a puncture through an interatrial septum of a heart of a patient. The method further includes inflating an inflatable balloon having a distal outer surface comprising an ablation region, bringing the distal outer surface of the inflatable balloon into contact with the interatrial septum proximal to the puncture, and applying ablation energy to the interatrial septum through the ablation region of the inflatable balloon, to create a multicuspid ablation pattern.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a perspective view of an example of a surgical apparatus according to one or more aspects of this disclosure.

FIG. 2A is a front view of the surgical apparatus of FIG. 1.

FIG. 2B is a front view of a surgical apparatus according to further aspects of this disclosure.

FIG. 3 is an illustration of a multicuspid ablation pattern according to one or more aspects of this disclosure.

FIG. 4 is a schematic illustration of an example of a surgical apparatus beginning an interatrial shunt procedure according to one or more aspects of this disclosure.

FIG. 5 is a schematic illustration of an example of a surgical apparatus contacting an interatrial septum according to one or more aspects of this disclosure.

FIG. 6 is a schematic illustration of an example of a surgical apparatus with a deflated balloon according to one or more aspects of this disclosure.

FIG. 7 is a schematic illustration of an example of a surgical apparatus advanced partially through an interatrial septum according to one or more aspects of this disclosure.

FIG. 8 is a schematic illustration of an example of a surgical apparatus dilating a puncture in an interatrial septum according to one or more aspects of this disclosure.

FIG. 9 is an illustration of tear propagation along a multicuspid ablation pattern according to one or more aspects of this disclosure.

FIG. 10 is an illustration of a tri-leaflet interatrial shunt according to one or more aspects of this disclosure.

FIG. 11 is a flow chart illustrating an example of a process of creating an interatrial shunt according to one or more aspects of this disclosure.

FIG. 12 is a perspective view of another example of a surgical apparatus according to one or more aspects of this disclosure.

FIG. 13 is a flow chart illustrating an example of a process of creating a multicuspid ablation pattern according to one or more aspects of this disclosure.

FIG. 14 is an illustration of a surgical apparatus using a cryogenic agent spray pattern according to one or more aspects of this disclosure.

FIG. 15 is a schematic illustration of a surgical system for creating an interatrial shunt according to some aspects of this disclosure.

### DETAILED DESCRIPTION

In various aspects, the present disclosure provides for a surgical apparatus that facilitates a controlled tearing or cleaving of ablated tissue to create a multicuspid, multi-leaflet interatrial shunt between the left and right atria of a patient's heart. This shunt alleviates elevated pressure in the left atrium caused by heart failure, by recycling the blood through the system back into the right atrium. In some aspects, scar tissue replaces the need for a stent for preventing tissue regrowth and to maintain the shunt. Having a shunt without a stent or similar mechanical implant reduces or eliminates the risk of stent failure. In further aspects, the disclosed surgical apparatus creates a multicuspid, multi-leaflet opening that exhibits improved behavior based on the differential pressure between the left and right atria, compared to a non-cuspid (e.g., circular) opening. Further, the disclosed surgical apparatus simplifies the procedure for creating a multi-leaflet shunt without requiring a mechanical cutter with blades.

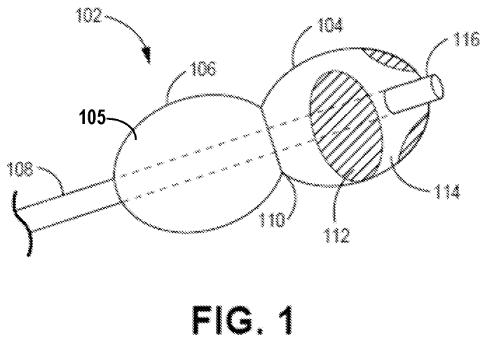

FIG. 15 is a schematic illustration of a surgical system 10 for creating an interatrial shunt according to some aspects of this disclosure. Surgical system 10 includes a device 12 configured to be coupled with a control unit 14. Device 12 includes an elongate body 18, e.g., a catheter and/or guidewire that can be steerable within a patient's circulatory system to a patient's heart. Device 12 further includes a proximal end 20 where a handle 24 is located, and a distal end 22 including a surgical apparatus 102, described below and illustrated in FIG. 1.

Handle 24 includes one or more interfaces with control unit 14, which is illustrated with an optional display 35. For example, handle 24 may be coupled with fluid supply reservoir 16 and a fluid recovery reservoir or scavenging system 40. Fluid supply reservoir 16 may include any suitable number of fluid supply reservoirs for any suitable fluids, including, for example, a cryogenic agent such as liquid nitrogen or liquid nitrous oxide, and a non-cryogenic agent such as air or gaseous nitrous oxide. According to an aspect of the disclosure, each reservoir of the fluid supply reservoirs 16 may separately deliver a respective agent via a lumen of the elongate body 18 to the surgical apparatus 102, as described further below. In some examples, the same medium may be used as a cryogenic agent and a non-cryogenic agent. For example, nitrous oxide may be introduced into the surgical apparatus 102 in a liquid form to deliver cryogenic energy to the surgical apparatus 102, and nitrous oxide may be introduced into the surgical apparatus 102 in a gaseous form to, e.g., inflate a balloon at the surgical apparatus 102. Control unit 14 further includes a processing circuit 33 for controlling electronic operations of the system 10, and an interface with a generator 37 for generating electromagnetic energy for, e.g., pulsed field ablation (PFA) and/or radio frequency (RF) ablation via surgical device 102. In the illustrated example, electromagnetic energy generator 37 includes an optional user interface 39. In another example (not illustrated), control unit 14 may further include an ultrasound generator for delivering ultrasound energy to surgical apparatus 102.

In some examples, the surgical apparatus 102 may perform ablation of tissue via electromagnetic or ultrasound energy. While in other examples, the surgical apparatus 102 may perform ablation of tissue via cryogenic energy. FIGS. 1-3, described below, illustrate an example aspect according to techniques of this disclosure that employ cryoablation of tissue.

FIG. 1 is a perspective view of a surgical apparatus 102 according to some aspects of this disclosure. Surgical apparatus 102 has an elongate tool body 108 that includes a dual-lobed inflatable balloon 105 having a distal lobe 104 and a proximal lobe 106. The elongate tool body 108 may include a catheter and/or a guidewire for guiding the surgical apparatus 102 through a patient's circulatory system. Elongate tool body 108 further includes one or more lumen and suitable structures to access the lumen at the proximal end 20 of the elongate tool body 108 (see FIG. 15) to introduce cryogenic and non-cryogenic agent into the inflatable balloon 105. That is, the elongate tool body 108 may be configured for advancing the surgical apparatus 102 into the heart, and the elongate tool body 108 may include one or more lumen operatively coupled to the control unit 14 (see FIG. 1). These lumen may include, for example, a first lumen for introducing a cryogenic agent into the surgical tool 102; a second lumen for introducing a non-cryogenic agent (e.g., an insulating agent) into the surgical tool 102; and in some examples, a further lumen for stowing the deflated balloon 105. The inflatable balloon 105 is configured to substantially conform to, or fit inside of a lumen when in a deflated state.

A waist 110 between the distal lobe 104 and proximal lobe 106 has a controlled diameter (e.g., a diameter between about 5 and 15 mm) for dilating an opening in the interatrial septum. The distal outer surface of the balloon 105 has at least one insulated region 112 and at least one ablation region (e.g., cooling region 114). When the balloon 105 is inflated, a cryogenic agent may be introduced to the balloon 105 via a lumen in the elongate tool body 108. When the cryogenic agent is introduced into the balloon 105, the cooling region 114 extracts heat from tissue with which it is in contact, while the insulated region 112 protects or insulates tissue from a cooling effect. A distal tip 116 of the elongate tool body 108 of the surgical apparatus 102 centers the cooling region 114 and assists in holding the surgical apparatus in position during a cryoablation procedure.

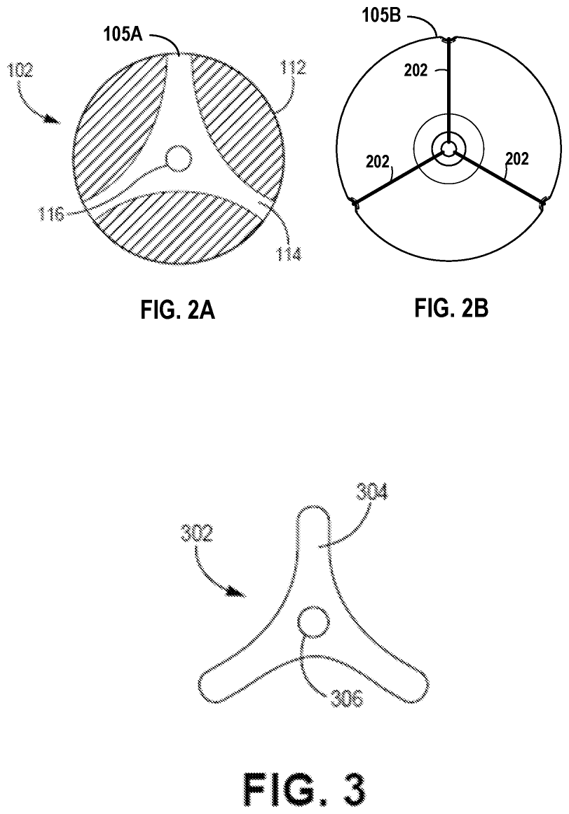

FIG. 2A is a front view of the distal end of the surgical apparatus 102, showing detail of the insulated region 112 and the cooling region 114 of the cryoablation balloon 105A. The cooling region 114 is configured for cooling tissue it is in contact with, causing cryoablation of the atrial septum in a preconfigured pattern.

FIG. 3 shows an example of an ablation pattern 302 in an interatrial septum. The ablation pattern 302 shows the pattern of destroyed or killed cells in the interatrial septum resulting from contact with the cooling region 114 of the cryoablation balloon when a cryogenic agent is introduced into the balloon 105. The illustrated ablation pattern matches the configuration of the cooling region 114, being a three-armed star shape that has three arms 304 extending radially outward from a puncture 306. Of course, the present disclosure is not limited to the three-armed star configuration illustrated in FIG. 3. In other examples, the ablation region may take any suitable shape for making a multicuspid shunt, such as a multi-armed star having any suitable number of arms, or a T-shape to create a two-cuspid shunt. The distal tip 116 of the elongate tool body 108 of the surgical apparatus 102 may engage with the puncture 306 as discussed below to secure and stabilize the surgical apparatus 102 in position for an ablation procedure.

As indicated above, according to some aspects of this disclosure, rather than cryoablation, ablation energy may be delivered to a surgical apparatus 102 via electromagnetic or ultrasound energy. For example, FIG. 2B illustrates an example balloon 105B having electrodes 202 for delivering electromagnetic energy to tissue to create a multicuspid ablation pattern according to an aspect of this disclosure. In this example, the balloon 105B comprises an ablation region that corresponds to the region of the electrodes 202. In some examples, electrodes 202 may deliver electromagnetic energy to cardiac tissue such as to create the multicuspid ablation pattern 302 described above and illustrated in FIG. 3. That is, in various aspects, the present disclosure is not limited to a particular technique for ablation, and in different examples, ablation energy may be delivered to create a multicuspid ablation pattern 302 using cryogenic fluid, using electromagnetic energy, using ultrasound energy, or using any other suitable form of ablation energy for performing irreversible electroporation of cardiac tissue in a multi-arm ablation pattern such as pattern 302.

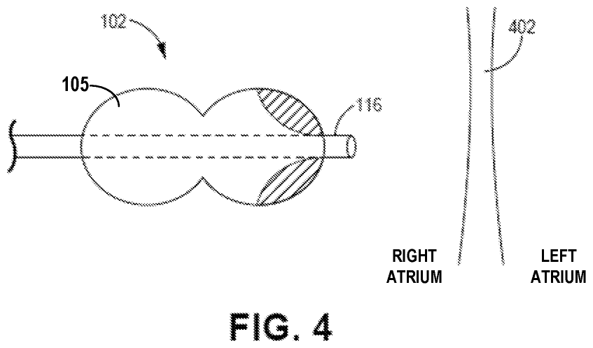

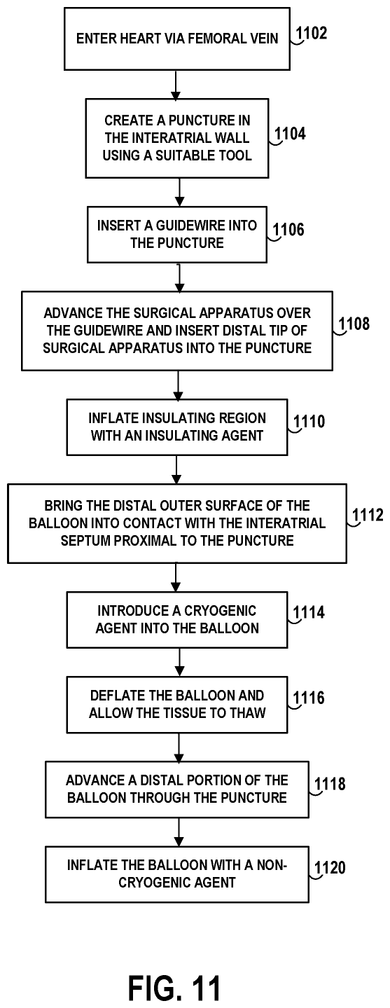

FIGS. 4-10 are described herein in conjunction with the flow chart of FIG. 11. These figures illustrate an exemplary process for creating a shunt in a patient's interatrial septum between the left and right atria according to some aspects of the present disclosure. As described below, a particular implementation may omit some or all illustrated features, and may not require some illustrated features to implement all embodiments. In some examples, the surgical apparatus illustrated in FIG. 1 may be configured to carry out the process illustrated in FIG. 11. In some examples, any suitable apparatus or means for carrying out the functions or algorithm described below may carry out the process illustrated in FIG. 11.

A surgical apparatus 102 may enter (1102) a patient's heart by being inserted into a suitable blood vessel such as the femoral vein and guided to the heart. FIG. 4 schematically illustrates a surgical apparatus 102 within a patient's heart in the right atrium and proximate to the interatrial septum 402. In the illustration of FIG. 4 the balloon 105 is inflated (e.g., with a non-cryogenic agent), but this need not necessarily be the case. That is, in some examples, the balloon 105 may remain deflated, and in some examples, stowed in a lumen until it is brought into contact with the interatrial septum 402.

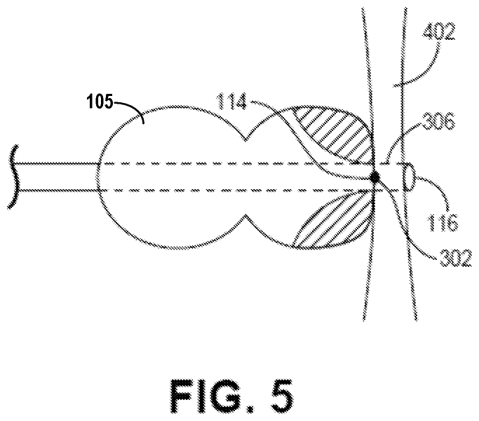

A puncture 306 may be created (1104) in the interatrial septum using any suitable tool, such as a needle, a knife, an electro-ablation blade, etc., which may be included as part of or advanced through the elongate tool body 108, or may be a separate element advanced through a catheter and/or over a guidewire. In some examples, a guidewire (not illustrated) of or advanced through the elongate tool body 108 of the surgical apparatus may be inserted (1106) into the puncture 306 for guiding and aligning the surgical apparatus 102 to the puncture 306. In some examples, a guidewire may be inserted into the puncture 306 by means other than elongate tool member 108, and elongate tool body 108 may be advanced to puncture 306 over the guidewire. In any case, as illustrated in FIG. 5, the distal tip 116 of the surgical apparatus 102 may be inserted (1108) into the puncture 306. For example, the elongate tool body 108 of the surgical apparatus 102 may be advanced over a guidewire to the location of the puncture 306 such that the distal tip 116 may be inserted into the puncture 306.

As discussed above, in some examples the balloon 105 may be inflated prior to being brought into contact with the interatrial septum 402. But in other examples, the balloon 105 may inflate after being brought into contact with the interatrial septum 402. Inflation of the balloon 105 may be performed by introducing a non-cryogenic agent such as air or gaseous nitrous oxide into an interior chamber of the balloon 105 via a lumen in elongate tool body 108 from the control unit 14 (see FIG. 1). The interior chamber of the balloon 105 may be defined by the inner surface of the balloon 105.

In some aspects of this disclosure, where the surgical apparatus is configured for cryoablation of tissue (e.g., as described in connection with FIG. 2A), the insulated region 112 may comprise a thicker portion of the cryoablation balloon. In some examples, the insulated region 112 may include a thermally insulating layer of plastic or other suitable polymer either on an interior surface or exterior surface of the balloon. In other examples, the insulated region 112 may be provided by way of an internal balloon coupled to an inner surface of the cryoablation balloon. That is, the balloon 105 may have a double-walled, two-layer structure, with one or more separately inflatable inner balloon(s) at the distal surface of the cryoablation balloon. That is, the balloon 105 may include an inner balloon and an outer balloon, and the surgical apparatus 102 may include a separate lumen for each of the inner and outer balloons for providing suitable inflating fluids or agents. In these examples, the insulated region 112 (e.g., the inner balloon) may be inflated (1110) with a suitable insulating agent. In this way, the insulating agent can be provided to certain portions of the balloon 105 (corresponding to the insulated region 112) between the balloon's walls or layers. Here, the insulating agent may be air, an insulating gas, liquid, or gel, or any suitable material for thermally insulating the insulated region 112.

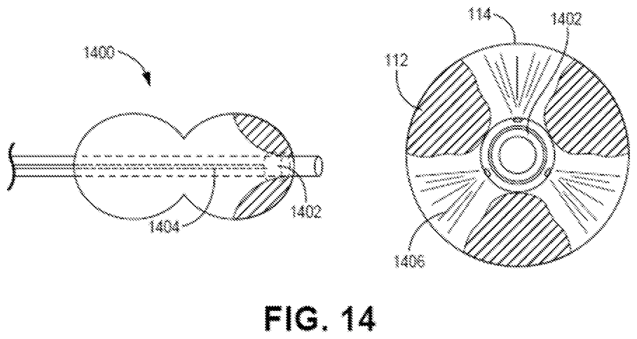

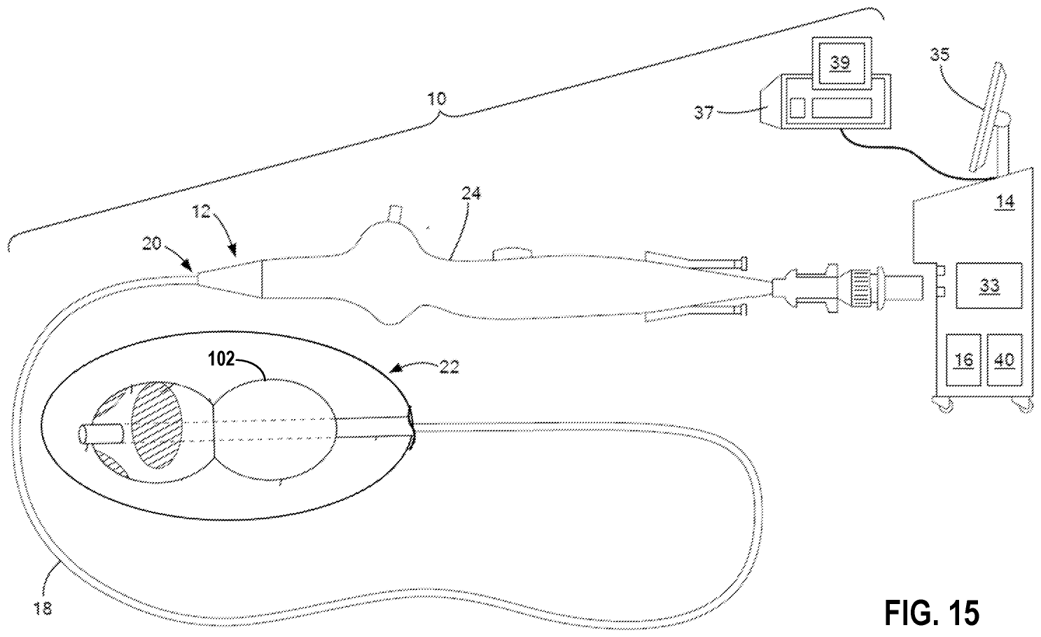

As illustrated in FIG. 5, the distal outer surface of the balloon 105 may be brought into contact (1112) with the interatrial septum 402 proximal to the puncture 306. The ablation region (e.g., the cooling region 114 in the cryoablation example illustrated in FIG. 5) contacts a contact region 302 of the interatrial septum 402 (i.e., the portion of the interatrial septum 402 that is in contact with the ablation region 114). Referring again to FIG. 3, the contact region 302 is the region where the ablation pattern will be located. That is, a cryogenic agent (e.g., nitrous oxide, carbon dioxide, nitrogen, or other near-critical gases) may be introduced (1114) into the balloon 105 via a lumen of the elongate tool body 108, causing the cooling region 114 to cool the contact region 302. The balloon 105 may receive the cryogenic agent in any suitable manner, including but not limited to spraying the cryogenic agent toward the interior surface of the cooling region(s) of the balloon 105 (see FIG. 14). Here, the insulated region 112 shields or insulates a portion of the interatrial septum 402 from the cooling effect of the cryogenic agent in the cryoablation balloon.

When the ablation region 114 is in contact with the contact region 302 of the interatrial septum 402, the distal tip 116 of the elongate tool body 108 is configured to align or stabilize the surgical apparatus 102. By applying a suitable pressure to press the elongate tool body 108 of the surgical apparatus 102 against the interatrial septum 402, the distal lobe 104 of the balloon 105 may conform to the surface of the interatrial septum 402, and/or the interatrial septum 402 may deform to wrap around a portion of the distal outer surface of the distal lobe of the cryoablation balloon.

According to some examples, a spray pattern of the cryogenic agent within the balloon may be adapted to spray the cryogenic agent on the cooling region 114 while avoiding spraying the cryogenic agent on the insulated region 112. For example, FIG. 14 shows an example of a balloon 1400 configured to employ a spray pattern for cryogenic agent according to some aspects of this disclosure. Balloon 1400 includes a coil 1402 having a plurality of injection ports or holes and a lumen 1404 for delivering the cryogenic agent. The coil is configured to spray the cryogenic agent 1406 on the cooling regions 114 while generally avoiding the insulated regions 112. Using a spray pattern in this fashion can improve the resolution of the ablation pattern, ensuring that cryoablation only occurs at the cooling regions 114.

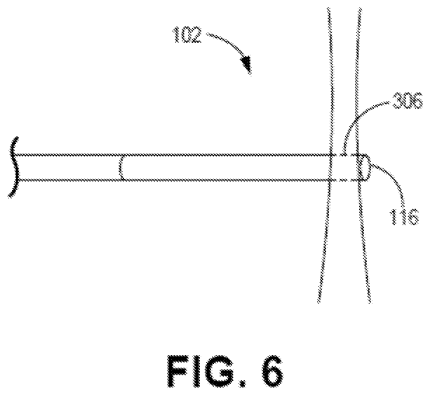

In some examples, a rotational fixation mechanism may be employed to prevent the balloon 105 from rotating, although other examples may omit such mechanism. After a suitable amount of time for ablation of the contact region 302 to take place (e.g., about 4 to 8 minutes in a cryoablation example), the surgical apparatus may deflate (1116) the interior chamber of the balloon(s) 105 and allow the tissue to thaw. For example, a thawing wait time may be established based on the temperature of the balloon 105, wherein the interior chamber of the balloon 105 automatically deflates when it reaches a suitable temperature (e.g., 20° C.). FIG. 6 shows the elongate tool body of the surgical apparatus 102 in a state with a deflated balloon, where the distal tip 116 of the surgical apparatus remains located in the puncture 306. In the illustration of FIG. 6, the balloon 105 may be stowed or retracted into a lumen, although this is not necessarily the case. In some examples, the elongate tool body 108 of the surgical apparatus may be withdrawn from the interatrial septum 402 while a guidewire (not shown) remains located in the puncture 306.

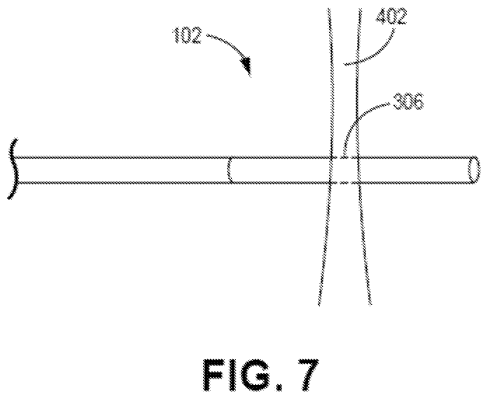

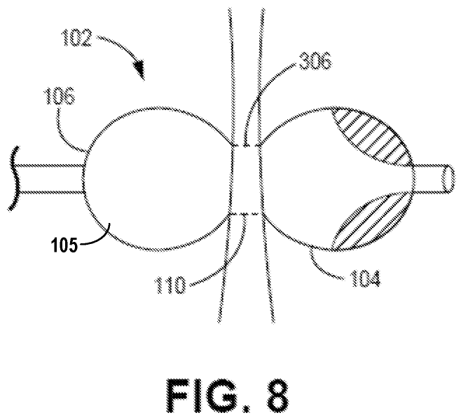

As shown in FIG. 7, the elongate tool body 108 of the surgical apparatus 102 with the deflated balloon(s) may advance (1118) through the puncture 306 into the left atrium chamber of the patient's heart. Here, a distal portion of the deflated balloon may cross the interatrial septum 402 and pass into the left atrium while a proximal portion of the deflated balloon may remain in the right atrium. Thus, as shown in FIG. 8, when the elongate tool body 108 of the surgical apparatus 102 inflates (1120) the interior chamber of its balloon 105 with a non-cryogenic agent (e.g., using air, gaseous nitrous oxide, or any other suitable medium), the distal lobe 104 and the proximal lobe 106 center the puncture 306 at the waist 110 of the balloon, and the waist 110 of the balloon expands or dilates the puncture 306 in a controlled manner, to a controlled diameter. For example, the waist 110 may have a smaller diameter (e.g., approximately 5-15 mm) than diameter of the distal lobe 104 and the proximal lobe 106 (which may be approximately 15-30 mm).

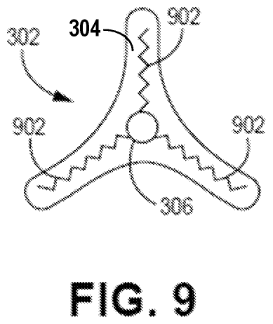

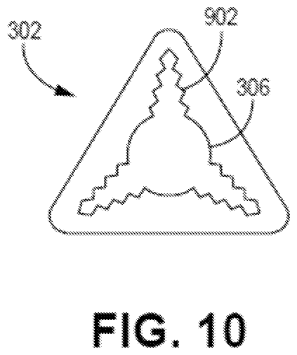

Because of the shaped ablation region 302, illustrated in FIG. 9, when the puncture 306 is dilated or expanded, the tissue is primed to tear or cleave 902 along the arms 304 of the ablation shape. That is, ablation of tissue in the interatrial septum creates stiffer tissue than untreated tissue. This stiffer tissue has a lower tensile strength than untreated tissue, such that when the opening 306 is dilated by the surgical apparatus 102, a tear 902 propagates along each of the arms 304 to create a multi-leaf, multicuspid opening as shown in FIG. 10.

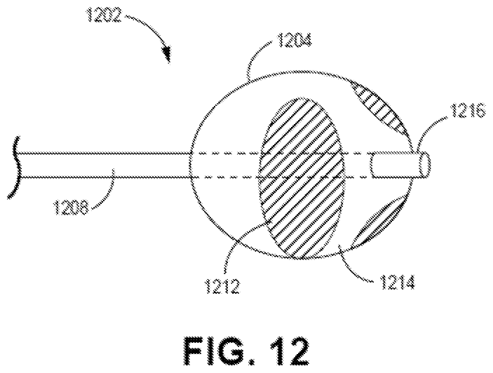

In the example described above, the elongate tool body 108 of the surgical apparatus 102 includes a dual-lobe balloon 105 having a distal lobe 104 and a proximal lobe 106. However, in other examples within the scope of this disclosure, e.g., as illustrated in FIG. 12, an elongate tool body 1208 of a surgical apparatus 1202 may employ a single-lobe balloon 1204. As in the previous cryoablation example, the balloon 1204 may have at least one insulated region 1212 and at least one ablation region (e.g., cooling region 1214) at a distal outer surface, with the ablation region shaped and configured to create a multicuspid ablation pattern proximal to a distal tip 1216 when the inflatable balloon 1204 receives ablation energy. And like the previous example, the balloon 1204 is configured to fit within or to conform to a lumen when in a deflated state. However, in this example, the outer diameter of the balloon 1204 is configured to have a size for controllably dilating a puncture in the interatrial septum. That is, in some aspects the balloon 1204 need not necessarily include a dual-lobe shape with a waist in between balloon lobes. Rather, the procedure may be carried out in substantially the same way with a single-lobe balloon 1204.

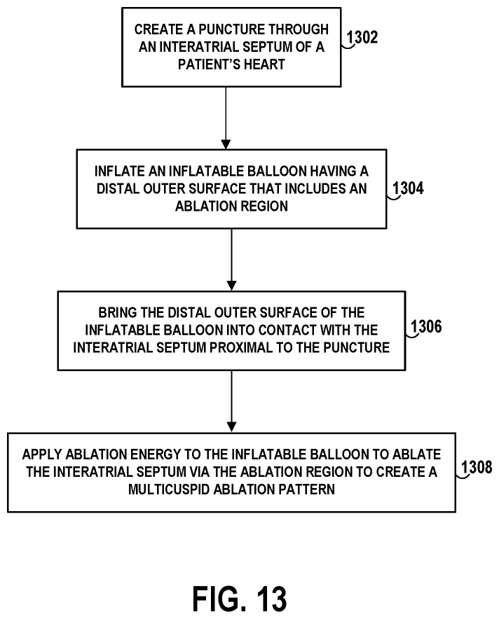

FIG. 13 is a flow chart illustrating a further exemplary process for creating a shunt in a patient's interatrial septum according to some aspects of this disclosure. As described below, a particular implementation may omit some or all illustrated features, and may not require some illustrated features to implement all embodiments. In some examples, the surgical apparatus 102 illustrated in FIGS. 1, 2A, 2B, 4-8, and 15, the surgical apparatus 1202 illustrated in FIG. 12, or the surgical apparatus 1400 illustrated in FIG. 14 may be configured to carry out the process illustrated in FIG. 13. In some examples, any suitable apparatus or means for carrying out the functions or algorithms described below may carry out the process illustrated in FIG. 13.

A surgical apparatus 102 may create (1302) a puncture 306 through an interatrial septum 402 of a patient's heart. The surgical apparatus 102 may use any suitable tool to create the puncture, such as a needle, a knife, an electro-ablation blade, etc. In some examples, the puncturing/cutting tool may be integrated to the surgical apparatus 102, while in other examples, the puncturing/cutting tool may be a separate tool. In some examples, the distal tip 116 of the surgical apparatus 102 may have a tapered profile providing for puncturing the interatrial septum.

An elongate tool body 108 of the surgical apparatus 102 may inflate (1304) an inflatable balloon. Here, the inflatable balloon includes a distal outer surface having an ablation region (e.g., a cooling region 114, and/or one or more electrodes 202, see FIGS. 2A and 2B). In a cryogenic example where the ablation region is a cooling region 114, the distal outer surface of the inflatable balloon may further include an insulated region 112. The insulated region 112 may be provided by a different (or thicker) material than the cooling region 114, or by one or more inner balloon(s) inflated with an insulating agent such as air.

The surgical apparatus 102 may bring (1306) the distal outer surface of the inflatable balloon into contact with the interatrial septum 402, proximal to the puncture 306. Here, the distal tip 116 of the surgical apparatus 102 may remain in the puncture 306 to center the surgical apparatus 102 and stabilize its position at the location where ablation will take place.

The surgical apparatus 102 may apply an ablation energy to the inflatable balloon 105 (1308). For example, the surgical apparatus 102 may apply a cryogenic agent to the interior surface of the cryoablation balloon. In this way, a cryoablation balloon extracts heat from the interatrial septum 402 through the cooling region 114, to create a multicuspid ablation pattern 302. In another example, the surgical apparatus 102 may apply electromagnetic energy to one or more electrodes on the distal outer surface of the inflatable balloon. In this way, an electromagnetic ablation electrode may ablate the interatrial septum 402 to create a multicuspid ablation pattern 302.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a surgical apparatus. The following examples are examples of systems, devices, and methods described herein.

Example 1: In some examples, a surgical apparatus includes an elongate tool body including at least one lumen and a distal portion, and an inflatable balloon on the distal portion, the inflatable balloon configured to be inflated via the lumen. The inflatable balloon includes a distal outer surface configured for contacting an interatrial septum of a heart of a patient, and an ablation region of the distal outer surface of the inflatable balloon configured for ablating the interatrial septum when the inflatable balloon receives the ablation energy. The ablation region is disposed on the distal outer surface of the inflatable balloon in a manner that the ablation region creates a multicuspid ablation pattern when the inflatable balloon receives the ablation energy.

Example 2: In some examples of the surgical apparatus of Example 1, the inflatable balloon is further configured for receiving a non-cryogenic agent via the lumen, wherein the elongate tool body further comprises a distal puncturing tip for puncturing the interatrial septum to create an opening, and wherein the inflatable balloon is configured to advance through the opening and to dilate the opening causing tissue to tear along the multicuspid ablation pattern.

Example 3: In some examples of the surgical apparatus of Examples 1 or 2, the inflatable balloon includes a dual-lobe balloon having a distal lobe and a proximal lobe with a waist in between the distal lobe and the proximal lobe, and wherein the waist is configured for dilating the opening causing the tissue to tear along the multicuspid ablation pattern.

Example 4: In some examples of the surgical apparatus of Example 3, a diameter of the distal lobe and the proximal lobe is between 15-30 mm, and a diameter of the waist is between 5-15 mm.

Example 5: In some examples of the surgical apparatus of Examples 1 to 4, the elongate tool body further includes a guidewire lumen configured for advancing over a guidewire.

Example 6: In some examples of the surgical apparatus of Examples 1 to 5, the ablation energy includes electromagnetic energy. The ablation region includes one or more electrodes shaped to create the multicuspid ablation pattern when the one or more electrodes are energized with the ablation energy.

Example 7: In some examples of the surgical apparatus of Examples 1 to 6, the multicuspid ablation pattern includes a multi-armed star.

Example 8: In some examples of the surgical apparatus of Examples 1 to 5, the ablation energy includes cryogenic energy. The apparatus further includes an insulated region on the distal outer surface of the inflatable balloon for insulating the interatrial septum from cooling from a cryogenic agent. Further, the ablation region and the insulated region are shaped to create the multicuspid ablation pattern.

Example 9: In some examples of the surgical apparatus of Example 8, the at least one lumen includes a first lumen configured to receive the cryogenic agent and a second lumen configured to receive an insulating agent, wherein the insulated region comprises an inner balloon separate from and coupled to an inner surface of the inflatable balloon. Here, the inner balloon is configured to inflate with the insulating agent for shielding the insulated region from the cryogenic agent.

Example 10: In some examples of the surgical apparatus of Examples 8 to 9, the insulated region and the cooling region are configured to give the cooling region a shape of a multi-armed star.

Example 11: In some examples of the surgical apparatus of Examples 8 to 10, the cooling region has a shape of a three-armed star.

Example 12: In some examples of the surgical apparatus of Examples 8 to 11, the surgical apparatus further includes a plurality of cryogenic agent injection ports within the inflatable balloon, configured to spray the cryogenic agent toward an inner surface of the cooling region.

Example 13: In some examples, a surgical apparatus includes an elongate tool body including at least one lumen and a distal portion. The elongate tool body includes a distal puncturing tip for puncturing an interatrial septum of a heart of a patient to create an opening; and an inflatable balloon configured to be inflated via the lumen of the elongate tool body. The inflatable balloon includes a distal outer surface configured for contacting the interatrial septum; and an ablation region of the distal outer surface of the inflatable balloon for ablating the interatrial septum when the balloon receives the ablation energy. The ablation region is disposed on the distal outer surface of the inflatable balloon in a manner that the ablation region creates a multicuspid ablation pattern when the inflatable balloon receives the ablation energy. The inflatable balloon is configured to advance through the opening and to dilate the opening causing tissue to tear along the multicuspid ablation pattern.

Example 14: In some examples of the surgical apparatus of Example 13, the inflatable balloon includes a dual-lobe balloon having a distal lobe and a proximal lobe with a waist in between the distal lobe and the proximal lobe, and wherein the waist is configured for dilating the opening causing the tissue to tear along the multicuspid ablation pattern.

Example 15: In some examples of the surgical apparatus of Examples 13 to 14, a diameter of the distal lobe and the proximal lobe is 15-30 mm, and a diameter of the waist is 5-15 mm.

Example 16: In some examples of the surgical apparatus of Examples 13 to 15, the ablation region has a shape of a multi-armed star.

Example 17: In some examples of the surgical apparatus of Examples 13 to 16, the cooling region has a shape of a three-armed star.

Example 18: In some examples of the surgical apparatus of Examples 13 to 17, the ablation energy includes cryogenic energy. The inflatable balloon includes an interior chamber for receiving a cryogenic agent, and an insulated region of the distal outer surface of the inflatable balloon for insulating the interatrial septum from cooling from the cryogenic agent.

Example 19: In some examples of the surgical apparatus of Examples 13 to 17, the ablation energy includes electromagnetic energy. The ablation region further includes one or more electrodes shaped to create the multicuspid ablation pattern when the one or more electrodes are energized with the ablation energy.

Example 20: In some examples, a surgical method includes creating a puncture through an interatrial septum of a heart of a patient; inflating an inflatable balloon having a distal outer surface comprising an ablation region; bringing the distal outer surface of the inflatable balloon into contact with the interatrial septum proximal to the puncture; and applying ablation energy to the interatrial septum through the ablation region of the inflatable balloon, to create a multicuspid ablation pattern.

Example 21: In some examples of the surgical method of Example 20, the surgical method further includes deflating the inflatable balloon; advancing a distal portion of the inflatable balloon through the puncture; and inflating the inflatable balloon with a non-cryogenic agent to cause the tissue to tear along the multicuspid ablation pattern.

Example 22: In some examples of the surgical method of Examples 20 to 21, advancing the distal portion of the inflatable balloon through the puncture includes advancing the inflatable balloon over a guidewire.

Example 23: In some examples of the surgical method of Examples 20 to 22, the method further includes inflating an inner balloon coupled to an inner surface of the inflatable balloon with an insulating agent.

Example 24: In some examples of the surgical method of Examples 20 to 23, the ablation energy includes electromagnetic energy. The ablation region includes one or more electrodes on a surface of the inflatable balloon, shaped to create the multicuspid ablation pattern when the one or more electrodes are energized with the ablation energy.

Example 25: In some examples of the surgical method of Examples 20 to 23, the ablation energy includes cryogenic energy. The ablation region includes a cooling region.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. An apparatus comprising:
an elongate tool body comprising at least one lumen and a distal portion; and
an inflatable balloon on the distal portion, the inflatable balloon configured to be inflated via the lumen;
wherein the inflatable balloon comprises:
a distal outer surface configured for contacting an interatrial septum of a heart of a patient;
an ablation region of the distal outer surface of the inflatable balloon configured for ablating the interatrial septum when the inflatable balloon receives the ablation energy;
wherein the ablation region is disposed on the distal outer surface of the inflatable balloon in a manner that the ablation region creates a multicuspid ablation pattern when the inflatable balloon receives the ablation energy.

2. The apparatus of claim 1, wherein the inflatable balloon is further configured for receiving a non-cryogenic agent via the lumen,
wherein the elongate tool body further comprises a distal puncturing tip for puncturing the interatrial septum to create an opening, and
wherein the inflatable balloon is configured to advance through the opening and to dilate the opening causing tissue to tear along the multicuspid ablation pattern.

3. The apparatus of claim 2, wherein the inflatable balloon comprises a dual-lobe balloon having a distal lobe and a proximal lobe with a waist in between the distal lobe and the proximal lobe, and wherein the waist is configured for dilating the opening causing the tissue to tear along the multicuspid ablation pattern.

4. The apparatus of claim 3, wherein a diameter of the distal lobe and the proximal lobe is between 15-30 mm, and wherein a diameter of the waist is between 5-15 mm.

5. The apparatus of claim 1, wherein the elongate tool body further comprises a guidewire lumen configured for advancing over a guidewire.

6. The apparatus of claim 1, wherein the ablation energy comprises electromagnetic energy, and
wherein the ablation region comprises one or more electrodes shaped to create the multicuspid ablation pattern when the one or more electrodes are energized with the ablation energy.

7. The apparatus of claim 6, wherein the multicuspid ablation pattern comprises a multi-armed star.

8. The apparatus of claim 1, further comprising an insulated region on the distal outer surface of the inflatable balloon for insulating the interatrial septum from cooling from a cryogenic agent,
wherein the ablation energy comprises cryogenic energy, and
wherein the ablation region and the insulated region are shaped to create the multicuspid ablation pattern.

9. The apparatus of claim 8, wherein the at least one lumen comprises a first lumen configured to receive the cryogenic agent and a second lumen configured to receive an insulating agent, wherein the insulated region comprises an inner balloon separate from and coupled to an inner surface of the inflatable balloon,
wherein the inner balloon is configured to inflate with the insulating agent for shielding the insulated region from the cryogenic agent.

10. The apparatus of claim 8, wherein the insulated region and the cooling region are configured to give the cooling region a shape of a multi-armed star.

11. The apparatus of claim 10, wherein the cooling region has a shape of a three-armed star.

12. The apparatus of claim 8, further comprising a plurality of cryogenic agent injection ports within the inflatable balloon, configured to spray the cryogenic agent toward an inner surface of the cooling region.

13. An apparatus comprising:
an elongate tool body comprising at least one lumen and a distal portion;
the elongate tool body comprising:
a distal puncturing tip for puncturing an interatrial septum of a heart of a patient to create an opening; and
an inflatable balloon configured to be inflated via the lumen of the elongate tool body,
wherein the inflatable balloon comprises:
a distal outer surface configured for contacting the interatrial septum;
an ablation region of the distal outer surface of the inflatable balloon for ablating the interatrial septum when the inflatable balloon receives the ablation energy,
wherein the ablation region is disposed on the distal outer surface of the inflatable balloon in a manner that the ablation region creates a multicuspid ablation pattern when the inflatable balloon receives the ablation energy, and
wherein the inflatable balloon is configured to advance through the opening and to dilate the opening causing tissue to tear along the multicuspid ablation pattern.

14. The apparatus of claim 13, wherein the inflatable balloon comprises a dual-lobe balloon having a distal lobe and a proximal lobe with a waist in between the distal lobe and the proximal lobe, and wherein the waist is configured for dilating the opening causing the tissue to tear along the multicuspid ablation pattern.

15. The apparatus of claim 14, wherein a diameter of the distal lobe and the proximal lobe is 15-30 mm, and wherein a diameter of the waist is 5-15 mm.

16. The apparatus of claim 13, wherein the ablation region has a shape of a multi-armed star.

17. The apparatus of claim 16, wherein the ablation region has a shape of a three-armed star.

18. The apparatus of claim 13, wherein:
the ablation energy comprises cryogenic energy, and
the inflatable balloon further comprises:
an interior chamber for receiving a cryogenic agent; and
an insulated region of the distal outer surface of the inflatable balloon for insulating the interatrial septum from cooling from the cryogenic agent.

19. The apparatus of claim 13, wherein:
the ablation energy comprises electromagnetic energy, and
the ablation region further comprises one or more electrodes shaped to create the multicuspid ablation pattern when the one or more electrodes are energized with the ablation energy.

20. A method comprising:
creating a puncture through an interatrial septum of a heart of a patient;
inflating an inflatable balloon having a distal outer surface comprising an ablation region;
bringing the distal outer surface of the inflatable balloon into contact with the interatrial septum proximal to the puncture; and
applying ablation energy to the interatrial septum through the ablation region of the inflatable balloon, to create a multicuspid ablation pattern.
